(19) United States
(12) Patent Application Publication
(10) Pub. No.: US 2016/0089490 A1
(43) Pub. Date: Mar. 31, 2016

(54) DRUG DELIVERY DEVICE INCLUDING ELECTROLYTIC PUMP

(71) Applicant: King Abdullah University of Science and Technology, Thuwal (SA)

(72) Inventors: Ian G. Foulds, Thuwal (SA); Ulrich Buttner, Thuwal (SA); Ying Yi, Thuwal (SA)

(21) Appl. No.: 14/867,393

(22) Filed: Sep. 28, 2015

Related U.S. Application Data
(60) Provisional application No. 62/057,428, filed on Sep. 30, 2014.

(51) Int. Cl. A61M 5/142

(52) U.S. Cl. CPC A61M 5/142; A61M 2005/14204; A61M 2202/06

(57) ABSTRACT

Systems and methods are provided for a drug delivery device and use of the device for drug delivery. In various aspects, the drug delivery device combines a “solid drug in reservoir” (SDR) system with an electrolytic pump. In various aspects an improved electrolytic pump is provided including, in particular, an improved electrolytic pump for use with a drug delivery device, for example an implantable drug delivery device. A catalytic reformer can be incorporated in a periodically pulsed electrolytic pump to provide stable pumping performance and reduced actuation cycle.

### CROSS-REFERENCE TO RELATED DOCUMENTS

This application claims the benefit of and priority to U.S. Provisional Patent Application No. 62/057,428 entitled “DRUG DELIVERY DEVICE INCLUDING ELECTROLYTIC PUMP”, filed on Sep. 30, 2014, which is expressly incorporated by reference as if fully set forth herein in its entirety.

### TECHNICAL FIELD

The present disclosure generally relates to drug delivery devices, in particular implantable drug delivery devices.

### BACKGROUND

Without timely and necessary treatments, some chronic diseases, such as glaucoma and diabetes, may lead to irreversible cell damage. Currently, drug therapies are the most widely used treatment for chronic diseases. Although extensive efforts have been devoted to the biomedical field and medical services, an effective method of drug delivery capable of directly targeting certain tissue regions within the body has still not been demonstrated.

Conventional drug delivery methods, such as oral ingestion, eye drops, and injections, do not exhibit targeted and controllable release. It was reported that less than 5% of the applied medication could penetrate physiological barriers, which means conventional drug delivery relies on a super overdose of medication. Although injections are a more targeted method, this type of drug delivery requires frequent treatments that cause discomfort and pain to patients.

In both conventional and immediate release methods, administrating a therapeutic drug concentration directly at the disease site is difficult. For many drug applications, drug delivery within a therapeutic range on an “on-demand” basis can be beneficial to patients; however an immediate release of drugs may cause concentrations above the therapeutic window to become toxic, and over time, the drug concentration could drop to inefficient levels (see FIG. 1).

In contrast to typical drug delivery systems, an implantable drug delivery device shows several critical advantages, such as high efficiency delivery, long term use, compliance with patients' demands and lack of frequent surgical or invasive requirements. Currently, non-mechanical pumping mechanisms have attracted growing interests. An electrolytic micro-pump, in particular, has potential for applications in the field of drug delivery systems, among the other options due to its low power consumption, high pumping efficiency, simple fabrication, easy operation and appropriate pumping force.

However, previous electrolytic pumps for drug delivery [1-2] have been operated using a liquid drug reservoir (LDR) approach, which means the drug dosing must be controlled by accurate delivery of extremely small fluid volumes. For electrolytic pumps this is complicated by the recombination of the gases back into water making long-term stable dosing difficult. The solid drug in reservoir (SDR) approach was recently developed using magnetic actuation to pump fluid in and out of a reservoir that is filled with a low solubility drug in solid form. This solves the stable dosing issue by allowing the drug to saturate the solution in the reservoir, which is then ejected to provide a single dose [3]. The reservoir can then refill and dissolve the next dose, which simplifies the delivery system's control to stable discrete doses. However, magnetic actuation complicates the system. Being orientation specific and short range, the location of the magnetic delivery system is limited.

Accordingly, there is a need to address the aforementioned deficiencies and inadequacies.

### SUMMARY

We provide a new drug delivery device, including in particular an implantable drug delivery device. In various aspects, our drug delivery device combines the simple control of a “solid drug in reservoir” (SDR) system with the lower power and flexibility of electrolytic pump systems. Our device provides a consistent drug release, during periodical actuation. We provide a new cyclic delivery model based on “solid drug in reservoir” and add a catalytic reformer to improve the bubble recombination rate, as well as to reduce the required power, which makes wireless power operation easily accomplished. Our catalytic reformer element improves the rate of the electrolysis-bubble generation and recombination, which means a pumping cycle time decrease. For chronic disease treatments, our pump exhibits great contributions, such as long time operation, on-demand and controllable drug release, short actuation cycles, stable pumping performance, and wireless power operations.

No previously reported works have combined a “solid drug in reservoir” (SDR) approach with an electrolytic pump. The present disclosure demonstrates detailed concepts, technologies and experimental data for a complete implantable drug delivery device involving an SDR based cyclic model and an electrolytic pump, showing feasibility of forming reproducible drug solution and long term disease treatments.

Moreover, in various aspects we provide an improved electrolytic pump, including in particular an improved electrolytic pump for use with a drug delivery device, for example an implantable drug delivery device. A catalytic reformer can be incorporated in a periodically pulsed electrolytic pump to provide stable pumping performance and reduced actuation cycle. In various aspects a sputtered platinum (Pt) coated nickel metal foam can be provided that not only accelerates the bubble recombination rate, but also reduces the required power, making wireless power operation accomplishable. The pump can be coupled with a reservoir configured to receive a drug in solid form for drug delivery.

The present disclosure provides a method to decrease the power consumption of the electrolytic pump, for the same volume of drug delivered, also increasing the recombination rate so that the cycle times can be increased.

In an embodiment, we provide a drug delivery device. The drug delivery device can comprise: a pump and a reservoir associated with the pump, the pump being an electrolytic pump including a chamber and a plurality of electrodes positioned within the chamber, the plurality of electrodes configured to be coupled to a power supply such that at least one of the electrodes is configured as an anode and at least another of the electrodes is configured as a cathode, the chamber being a sealed chamber configured to hold an electrolyte, the chamber having a side including a flexible impermeable membrane, the pump further including a reformer positioned within the chamber, the reformer configured to recombine gases generated by the electrolytic pump from an electrolyte contained within the chamber, the reservoir positioned in association with the membrane of the pump, and the reservoir configured to receive a drug in solid form and having an orifice configured to allow bodily fluid to pass through the orifice into the reservoir and to allow bodily fluid including the drug to be expelled from the reservoir upon deflection of the membrane.

In an embodiment, we provide a method of drug delivery. The method can comprise the steps of: providing a pump and a reservoir associated with the pump, the pump being an electrolytic pump including a chamber and a plurality of electrodes positioned within the chamber, the plurality of electrodes configured to be coupled to a power supply such that at least one of the electrodes is configured as an anode and at least another of the electrodes is configured as a cathode, the chamber being a sealed chamber holding an electrolyte, the chamber having a side including a flexible impermeable membrane, the pump further including a reformer positioned within the chamber, the reformer configured to recombine gases generated by the electrolytic pump from the electrolyte contained within the chamber, the reservoir positioned in association with the membrane of the pump, and the reservoir configured to receive a drug in solid form and having an orifice configured to allow bodily fluid to pass through the orifice into the reservoir and to allow bodily fluid including the drug to be expelled from the reservoir upon deflection of the membrane, placing a drug in solid form in the reservoir; using the pump to draw a bodily fluid into the reservoir and dissolve at least some of the solid drug in the bodily fluid; applying a voltage to the anode electrode and the cathode electrode to thereby generate gas from the electrolyte and generate an increase in pressure within the chamber of the pump causing the membrane to expand and put pressure on the reservoir and causing bodily fluid including the dissolved drug to be expelled from the reservoir; turning off the applied voltage to the anode and cathode electrodes causing a recombination by the reformer of the gas into the electrolyte thereby causing decrease in pressure within the chamber of the pump causing a decrease in the expansion of the membrane and a resultant pressure drop within the reservoir causing bodily fluid to be drawn into the reservoir; and repeating one or more cycles of applying voltage and turning off the applied voltage to the anode and cathode electrodes.

In any one or more aspects of any one or more of the embodiments the plurality of electrodes can comprise an array of electrodes. The array of electrodes can be formed by a sputtering technique and patterned on a substrate. The electrodes can be coated with an ionomer that has cationic conductive properties. The electrodes can be platinum electrodes. The reformer can be a catalytic reformer. The reformer can be a mesh scaffold. The reformer can be a platinum-coated mesh scaffold. The electrolyte can include water. The scaffold can be selected from the group consisting of catalytic metals and inert materials. The catalytic metals can include platinum, or nickel or both. The inert materials can include carbon fiber mesh or a polymer porous mesh or both. The reformer can be fabricated by sputtering, depositing or electroplating platinum onto a scaffold.

Other systems, methods, features, and advantages of the present disclosure for our drug delivery device will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings like reference numerals designate corresponding parts throughout the several views.

FIG. 1 is an Illustration depicting a therapeutic window and different releasing profiles for a drug delivery.

FIG. 2 depicts (A) a 3D sketch of an embodiment of an electrolytic actuator showing the major components, and (B) an embodiment of an electrolytic chamber assembled into a drug delivery device of the present disclosure.

FIG. 3 depicts a cross-sectional view of an embodiment of a drug delivery device of the present disclosure showing major system components.

FIG. 4 depicts (A) a micrograph of a 400 nm thick Pt electrode layout showing element width and spacing of both 100 μm, (B) a micro-scope of a Pt-coated nickel mesh, and (C) a photograph of a pumping chamber with a Pt mesh and Nafion coated electrode of an embodiment of an electrolytic actuator or pump of the present disclosure.

FIG. 5 is a schematic illustration of an embodiment of an SDR-based drug delivery device and its cyclic operation of the present disclosure.

FIG. 6 depicts a micro-scope of a Pt-coated nickel mesh fabricated by (A) sputtering, and (B) electroplating.

FIG. 7 is (A) an SEM image of overall Pt coated carbon fiber mesh, wherein the magnified region shows the edge from (B) top view and (C) cross-sectional view, and (D) a corresponding prototype of Pt coated carbon fiber mesh.

FIG. 8 depicts photographs of an experimental apparatus and structure of a drug delivery device of the present disclosure, wherein (A) depicts an assembled electrolytic pump for experimental measurements, (B) is a photograph of a laser drilled-drug reservoir from PMMA board, and (C) is a photograph of a pumping chamber with a Pt coated mesh and Nafion coated electrode.

FIG. 9 is a photograph of a Ti/Pt electrode design, wherein (A) shows the electrodes fabricated on silicon with Nafion coating, and (B) is a microscope of the interdigitated electrode layout showing element width and spacing.

FIG. 10 depicts electrolytic pump flow rate versus applied power for a platinum coated mesh reformer and a mesh reformer without platinum coating.

FIG. 11 depicts gas recombination rate comparisons for electrolytic pumps with and without a platinum mesh reformer with a power of 4.6 mW applied until the membrane achieved a maximum displacement and then turned off.

FIG. 12 is a photograph of an experimental setup and figure of a drug delivery device, including an electrolytic pump, of the present disclosure.

FIG. 13 depicts pumping performance comparisons between three different pump versions, wherein (A) shows calculated pump efficiency results versus current density for the pump with and without a Pt coated mesh, and (B) shows electrolytic pump flow rate versus applied power with the Pt-coated nickel metal foam and without the Pt-coated nickel metal foam.

FIG. 14 depicts power controlled flow rates for an electrolytic pump with and without a catalytic reformer (Mean±SE, n=3).

FIG. 15 depicts gas recombination rate comparisons for electrolytic pumps with and without catalytic reformer, wherein a power of 3.7 mW was applied until the membrane achieved a maximum displacement and then turned off.

FIG. 16 depicts real-time membrane displacement of an electrolytic pump with and without catalytic reformer under same experimental conditions, wherein a power of 4 mW was turned on and then turned off.

FIG. 17 depicts cyclic membrane displacement operated with different applied power showing stable behaviors of electrolysis bubble generation and recombination.

FIG. 18 depicts membrane displacement operated with different actuation frequencies showing the relationship between non-actuation interval and released volume for cyclic mode.

FIG. 19 depicts periodical pumping pulses showing behaviors of electrolytic bubble and gas recombination.

FIG. 20 depicts an experimental apparatus for drug delivery, and a side view of the device depicting release of solvent blue into external solution upon electrolysis actuation (see inset).

FIG. 21 depicts a dose of released drug per pulse under an applied power of 1 mW.

FIG. 22 depicts intermittent release of solvent blue 38 from the pump in the external reservoir by applied power of 4 mW, wherein solvent blue 38 was cumulatively pumped by a series of actuations. After each pumping a period of delivery is followed with no actuation mode. Release rate of 4.06 ug±0.26 ug per each actuation interval of 30 seconds indicates the feasibility and stability of an embodiment of our present device (see inset).

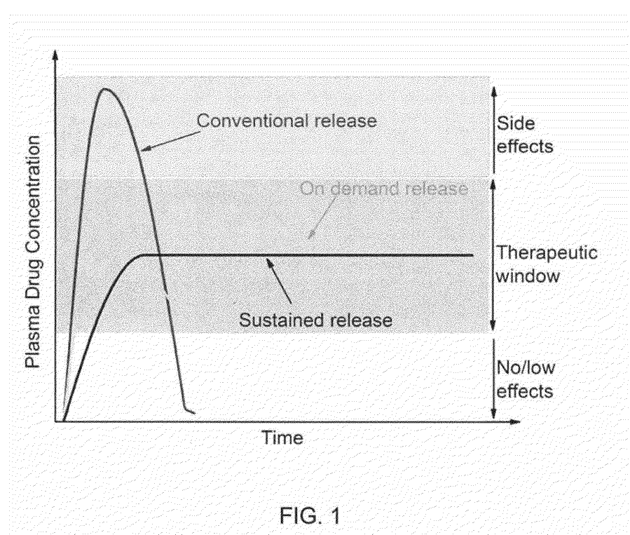

### DETAILED DESCRIPTION

Described below are various embodiments of the present systems and methods for a drug delivery device and use of the device for drug delivery. Although particular embodiments are described, those embodiments are mere exemplary implementations of the systems and methods. One skilled in the art will recognize other embodiments are possible. All such embodiments are intended to fall within the scope of this disclosure. Moreover, all references cited herein are intended to be and are hereby incorporated by reference into this disclosure as if fully set forth herein. While the disclosure will now be described in reference to the above drawings, there is no intent to limit it to the embodiment or embodiments disclosed herein. On the contrary, the intent is to cover all alternatives, modifications and equivalents included within the spirit and scope of the disclosure.

Implantable drug delivery technologies are important in the field of biomedical engineering. They can deliver a drug solution to injured issue directly and avoid repeated surgeries. An example is that diabetic patients often need insulin rejections. Once the drug delivery device is transplanted into a human body, drug delivery can be operated according to a patient's instructions for a long use, reducing the medical cost and repeated pain. Currently, implantable drug delivery related devices exhibit the future trend of biomedical engineering. They not only attract the research interests of the academic societies, but also provide benefits to the pharmaceutical factory and biomedical devices manufacturers.

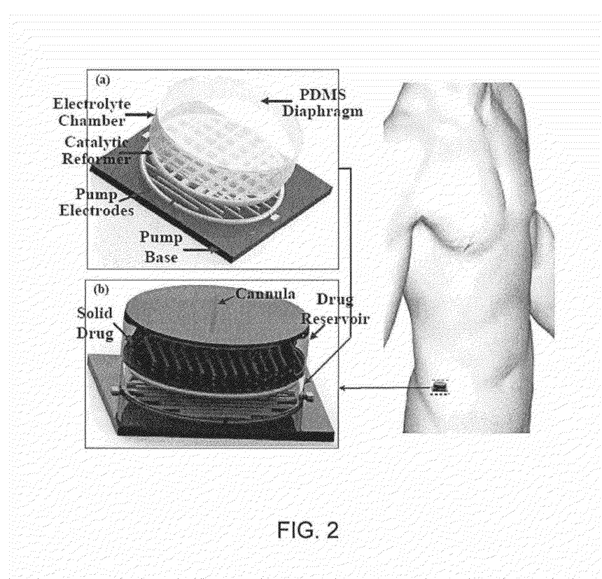

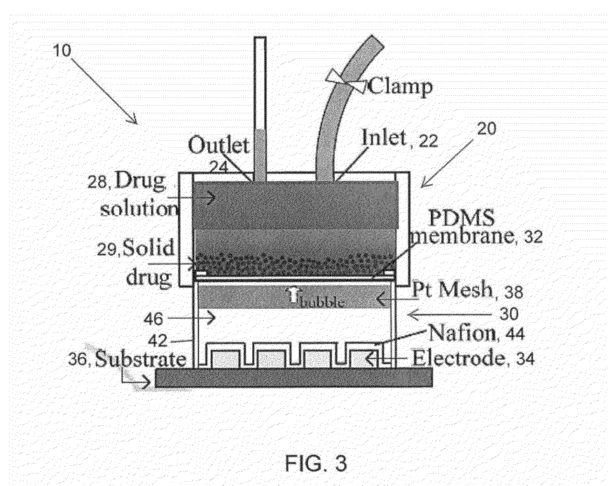

The present disclosure is directed to drug delivery device. In various aspects it provides an implantable drug delivery device or system. The FIGS. 2 and 3 present an embodiment of the present design of a drug delivery system. In various aspects the system can deliver pulsed doses of a drug that can be provided to the device in solid form. The drug can be a low solubility drug.

The system 10 can include a reservoir 20 and a pump 30. The reservoir 20 can have an inlet 22 and an outlet 24. A drug in solid form 29 can be placed in the reservoir 20. The system can pull bodily fluid through the inlet 22 into the reservoir 20. The reservoir can include the drug in solid form 29, allowing the drug to dissolve in the bodily fluid pulled into the reservoir 20. The pump 30 can then eject the drug laced bodily fluid 28 through the outlet 24 back into the tissue of the body where the drug solution 28 can be released into the body. The inlet 22 and the outlet 24 can be provided by a cannula 26. For easy prototype testing, the cannula 26 can be replaced by an inlet 22 and outlet tube 24 which can be mechanically clamped (see FIG. 3).

The pump 30 can be a resonant power transfer system, for example an electrolytic pump, and a cyclical actuation of the drug delivery system can be performed using the pump. By periodical pulsed pumping a stable and constant drug release can be accomplished, so that the effect of the delivered drug is controllable.

The electrolytic pump 30 can include: a membrane 32, a plurality of electrodes 34, and a chamber 42. The membrane 32 can be a flat membrane and can be comprised of polydimethylsiloxane (PDMS), though it can have other configurations and be made of other materials (for example parylene). The membrane 32, however, should be a flexible impermeable material to allow flexing of the material to provide the pumping action required.

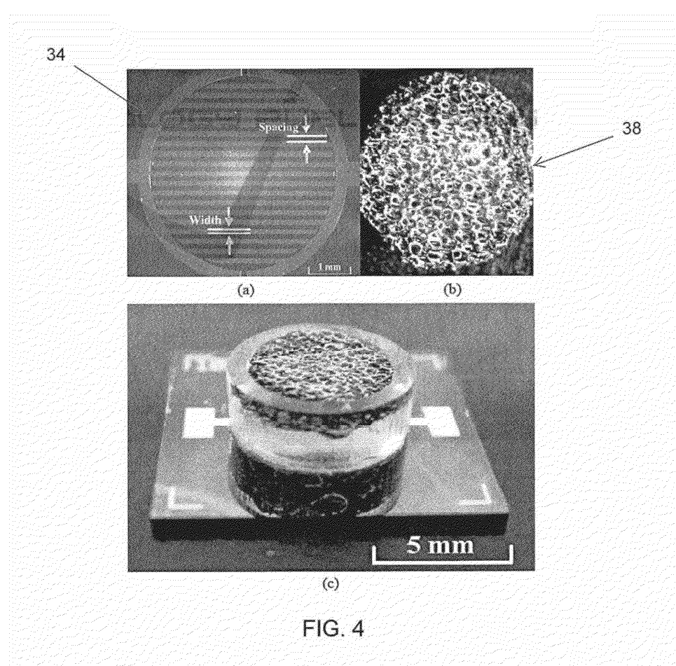

The plurality of electrodes 34 can be a pair of electrodes or preferably an array of a plurality of electrodes. An electrical power supply or power source (not shown), for example a DC power supply, can be coupled to the electrodes 34. In an aspect the electrodes 34 can be one or more platinum (Pt) electrodes. An array of electrodes can be fabricated, for example by a sputtering technique and then patterned on a base 36, for example a silicon wafer. An exemplary corresponding electrode array design is shown in FIG. 4A, though other array designs can be employed. The electrodes 34 can be contained within the chamber 42. The chamber 42 can be filled with water 46 for example, deionized (DI) water. The electrodes 34 can be immersed in a deionized (DI) water filled sealed pumping chamber 42 before assembling the other components. Other suitable materials for the electrodes include silver, gold, tantalum, graphite, vitreous carbon and other conductive materials that are relatively inert with respect to the water a liquid to be held in the chamber 42.

A reformer 38 can also be provided in the chamber 42 in the water 46. The reformer 38 can be a metal mesh material. For example, in an embodiment platinum was sputtered onto the surface of a nickel mesh forming a platinum coated nickel mesh reformer. Because the direction of sputtering is vertical, the horizontal aspect of the nickel mesh was not covered by platinum. When the mesh 38 was immersed into the water 46, both platinum and nickel were exposed to the water. A corresponding micrograph of an exemplary platinum coated nickel mesh reformer 38 is shown in FIG. 4B.

FIG. 4C illustrates a prototype of a pump 30 including a pumping chamber 42. The electrodes 34 can be coated with a solid polymer material 44, such as Nafion or other ionomer that has cationic conductive properties, to get a faster electrolysis-based bubble generation rate [4].

The reservoir 20 can be positioned adjacent and in contact with the pump 30 so the pumping action of the pump 30, described below, can act directly upon the reservoir. The pumping chamber 42 can be separated from the drug reservoir 20 by the membrane 32 to avoid electrochemical interaction with drug fluids 28 in the reservoir 20, but also to allow the membrane to act directly on the reservoir 20.

The electrical power supply can be coupled to the electrodes such that a positive charge is applied to at least one electrode and a negative charge is applied to at least one other electrode, thereby creating an anode and a cathode, respectively. When a voltage is then applied to the electrodes 34, electrolysis reactions occur in the water 46 in chamber 42 resulting in hydrogen (H₂) and oxygen (O₂) gas bubble generation. If the process occurs in pure water, H⁺ cations will generally accumulate at the anode and OH⁻ anions will generally accumulate at the cathode. Electrolysis of pure water can require application of excess energy in the form of over potential to overcome various activation barriers. Without excess energy the electrolysis of pure water can occur very slowly. The electrolysis rate can be increased by adding an ionic electrolyte to the water 46. In various aspects, the voltage applied to the electrodes can be a DC voltage and the amount of voltage to be applied to the electrodes can range from about 1.23V to about 12 V.

The gas bubbles generated can cause gas expansion within the chamber 42 pushing the membrane 32 upwards or outwardly against the reservoir 20 to in turn push dissolved drug solution 28 out of the reservoir outlet 24 or cannula 26. Power can be applied until the desired volume of fluid including dissolved drug solution 28 is delivered into the body, after which the power to the electrodes 34 can be turned off and the pressure in the electrolytic reservoir 20 begins decreasing due to the recombination of H₂ and O₂ within chamber 42.

The rate of the recombination can be improved by the inclusion of reformer 38 within the chamber 42. The recombination causes a pressure drop within the chamber 42 causing the membrane 32 to move downward drawing fresh bodily fluids into the drug reservoir 20 to dissolve more of the remaining solid drug 29. Power can be periodically turned on and off to the electrodes 34 of the pump 30 so that the dissolved drug solution 28 can be delivered into the body using this cyclical actuation.

### The Operation Mechanism

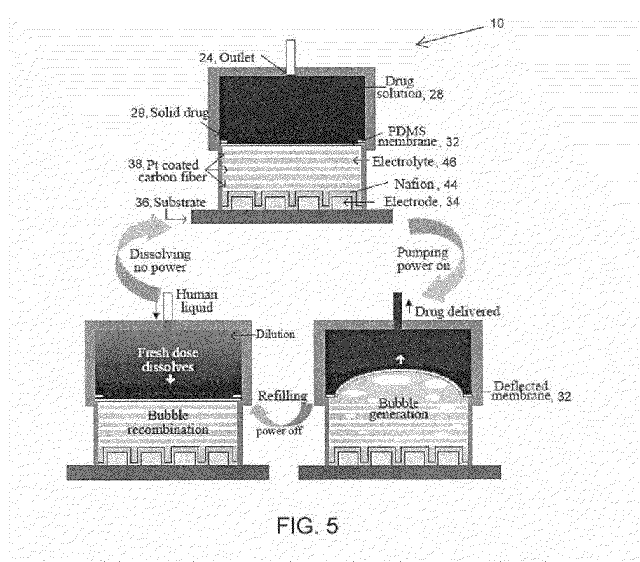

In an embodiment, assembly of the pump 30 can involve first filling the chamber 42 with DI water 46 and joining it to a pump base 36 to seal the electrolysis reaction chamber 42. Voltage can be applied to the electrodes 34, resulting in an electrochemical reaction electrolyzing the DI water 46 in the chamber 42 causing the production of gas bubbles, in particular hydrogen, H₂, and oxygen, O₂, gas bubbles. The gas generation inside the pumping chamber 42 provides a force upon the membrane 32, causing deflection of the membrane outwardly from the chamber 42. The outward deflection of membrane 32 places pressure against reservoir 20, so that dissolved drug 28 stored in the reservoir 20 is pushed out of the outlet 24 or cannula 26 of the reservoir 20 and can be delivered into the human body (see FIG. 5).

When the power is turned off, the pressure decrease inside the electrolyte chamber 42 can be correlated strongly with recombination of hydrogen (H₂) and oxygen (O₂) within the chamber 42 due to the properties of the reformer 38. The decrease in pressure causes the deflected membrane 32 to return to its original un-deflected position, resulting in bodily fluid being drawn into the drug reservoir 20 to dissolve additional solid drug 29 contained therein.

In a preferred embodiment, the reformer 38 is a platinum coated mesh scaffold. The reformer 38 can be comprised of a catalytic material, such as platinum, that can combine the generated gas into water. The mesh scaffold 38 including catalytic metal, such as nickel, can perform as a catalytic reformer to achieve a higher or faster bubble generation rate and recombination rate which can reduce the pumping cycle time. Other suitable scaffold materials include inert materials that not interact with the electrolyte, standing for a long term use, such as carbon fiber mesh.

In one or more aspects, the electrodes 34 are formed as an array comprised of a catalytic material such as platinum, for example fabricated by a sputtering technique and then patterned on a silicon wafer. The dimensional parameters can be 100 μm in width with 100 μm spacing and a 300 nm-400 nm height. The diameter of the electrode can be 5 mm. Other sizes and dimensions can be used however. The use of catalytic material in both the electrodes 34 and the reformer 38 can further aid in reducing the pumping cycle time and, thus, better control of delivery of drug solution 28 from the reservoir 20 into the body.

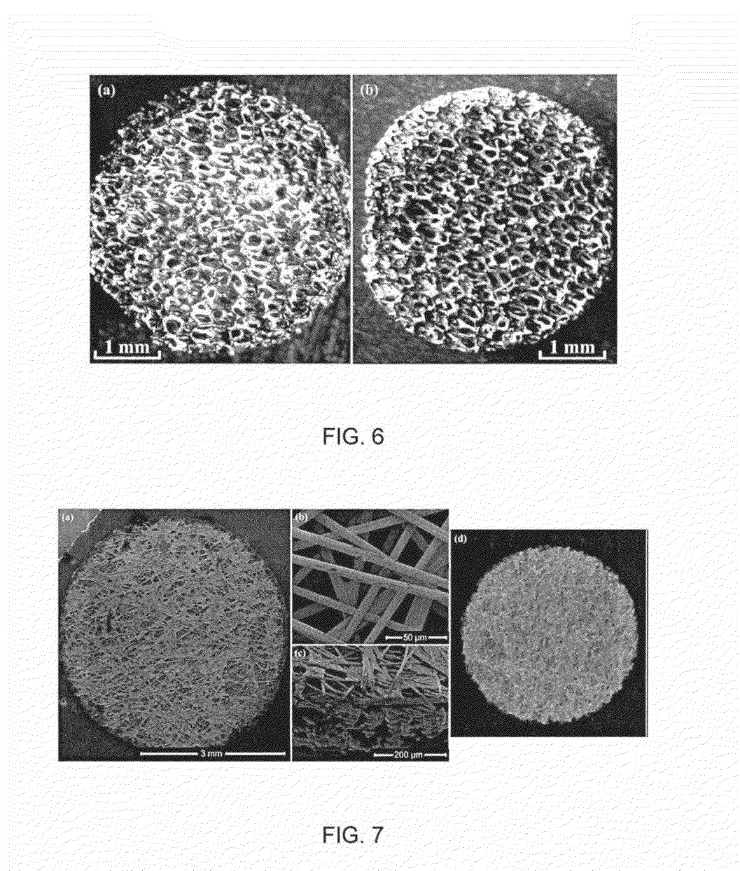

To fabricate a mesh scaffold, at least two methods can be applied: sputtering and electroplating. We show three different examples below. One is a sputtered Pt coated Nickel metal foam. Another is an electroplated Pt coated Nickel metal foam, and a third is a sputtered Pt coated Carbon fiber mesh. The corresponding prototypes of Pt coated nickel metal foams (by sputtering and electroplating techniques) are shown in FIGS. 6A and 6B. An exemplary sputtered Pt coated carbon fiber mesh, the prototype and SEM images (FIGS. 7A-7D) show the structure details.

Additional details of our systems and methods for a drug delivery device and use of the drug delivery device for drug delivery can be found in “TOWARDS AN IMPLANTABLE PULSED MODE ELECTROLYTIC DRUG DELIVERY SYSTEM”, MicroTas 2013, October, 2013, Germany, pp: 527-529, and “AN IMPROVED ELECTROLYTIC PUMP FOR POTENTIAL DRUG DELIVERY APPLICATIONS”, Biodevices 2014, March, 2014, France, which are expressly incorporated by reference as if fully set forth herein in their entireties.

Where a range of values is provided, it is understood that each intervening value, to the tenth of the unit of the lower limit unless the context dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both are also included in the disclosure.

Ratios, concentrations, amounts, and other numerical data may be expressed in a range format. It is to be understood that such a range format is used for convenience and brevity, and should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figure of the numerical value. In addition, the phrase “about ‘x’ to ‘y’” includes “about ‘x’ to about ‘y’”.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

All publications and patents cited in this specification are incorporated by reference as if each individual publication or patent were specifically and individually indicated to be incorporated by reference and are incorporated by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could differ from the actual publication dates that may need to be independently confirmed.

As will be apparent to those of skill in the art upon reading this disclosure, each of the individual embodiments described and illustrated has discrete components and features which may be readily separated from or combined with the features of any of the other several embodiments without departing from the scope or spirit of the present disclosure. Any recited method can be carried out in the order of events recited or in any other order logically possible.

### Example

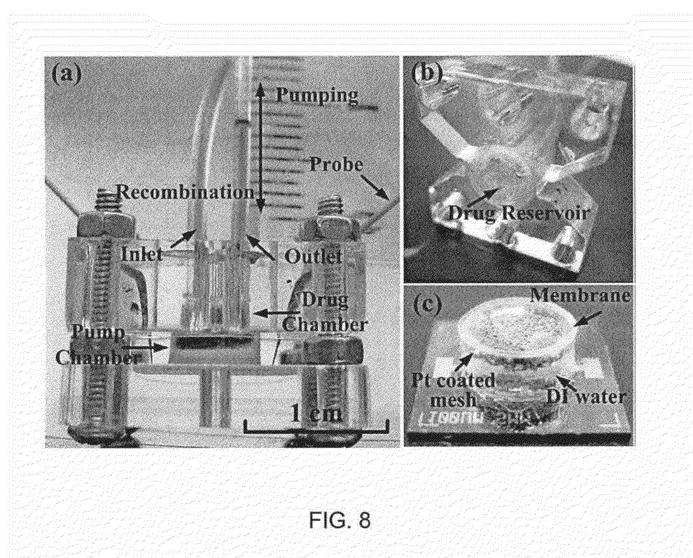

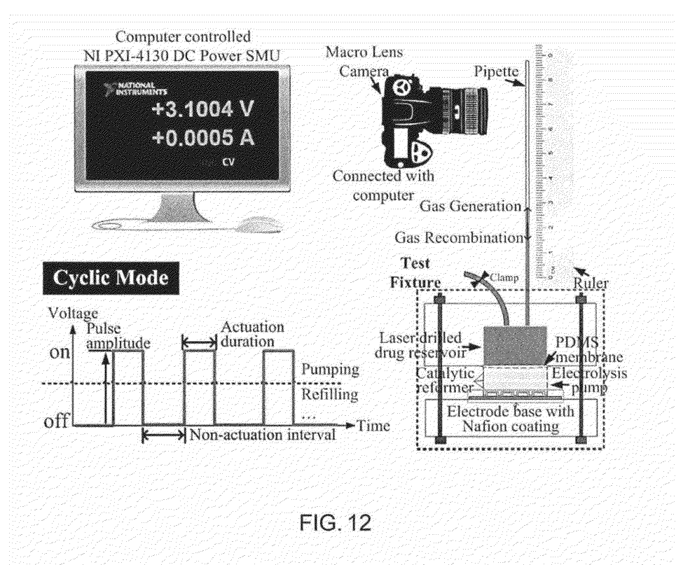

An embodiment our proposed drug delivery system, including an electrolytic pump, was assembled and measured using the test fixture (as an example, sputtered Pt coated nickel metal foam was used here) as shown in FIG. 8. The size of the holder is 2 cm: 2 cm: 2 cm (length: width: height). In prospective drug delivery applications, the pump could be permanently bonded allowing the size of the structure to be significantly reduced. Two probes are used to apply DC voltage to the electrodes. A digital camera was placed in front of the setup to record the displacement rate of the pump (see FIG. 12). In the experiment, Nafion was uniformly spin-coated onto electrodes to form a 235 nm thin film because it is capable of preventing bubble occlusion on the surface of the Pt electrode and improving diffusion of gases away from the catalyst surface [6].

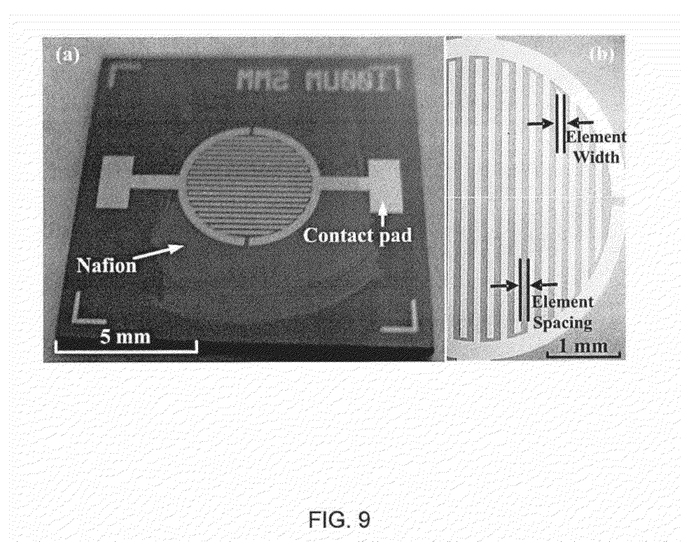

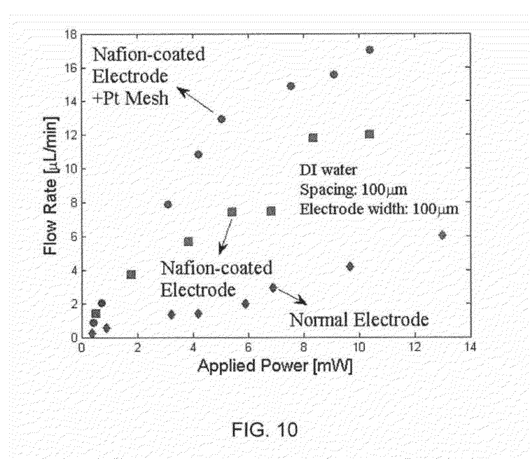

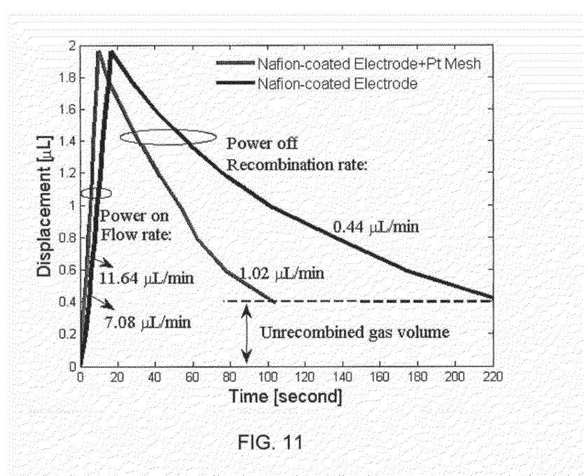

Electrode dimensions (100 μm element width and spacing)[2] and Nafion coating [4], as shown in FIG. 9, had been previously analyzed for achieving a higher pumping efficiency, as shown in FIG. 10. Based on the same experimental conditions and electrodes, we added a Pt mesh reformer as shown in FIG. 4B, and obtained a higher flow rate than previous works (see FIG. 10) as well as a faster recombination rate (see FIG. 11), because Pt mesh increases the contact area between the catalyst and electrolyte (e.g., water), improving catalytic reactions. Because the concentration of drug dose can be kept stable during each delivery [5], a faster bubble generation rate and recombination rate that reduce each period of pumping allow delivering a consistent and high drug volume within a short pumping duration. Most importantly the Pt mesh reformer used in the electrolytic pump requires less power (several mW) to achieve the same flow rate level compared to the original electrolytic pump, which makes the integration of wireless power transfer techniques [5] and drug delivery systems feasible.

FIG. 12 indicates the schematic diagrams of an experimental setup for the characterization and cyclic operation of our proposed electrolysis-bubble actuated pump. The DC power source measure unit (SMU) can supply the power to the pump via the probes connected the contact pads of the electrodes, then fluid climbs up due to the generation of electrolysis bubble. While power is removed, the fluid drops down due the recombination of the gases in the electrolysis chamber. The camera was used for real-time flow tracking.

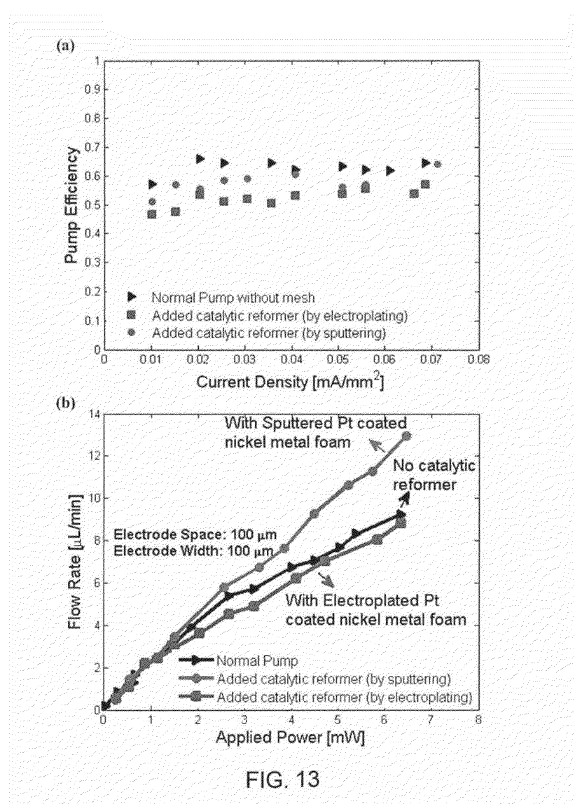

FIG. 13 shows a comparison of the pumping performance of three different pump versions run under the same experimental conditions: one with the aforementioned electrode dimensions and a Nafion coating; one with an added sputtered Pt coated metal foam; and one with an added electroplated Pt coated metal foam. Pumping efficiencies were lowered with the catalytic reformer (FIG. 13(a)). The results indicate that the added Pt-coated metal foam increases the contact area between the Pt and formed gas, and it decreases pumping efficiency due to the competitive bubble recombination catalyzed by Pt, even during the pumping phase. As the electroplated Pt-coated metal foam has a larger Pt contact area than the sputtered Pt-coated one, it provides a lower pumping efficiency.

The electrolytic reaction is strongly related to the applied current: a higher current induces faster electrolysis bubble generation. However, when the Ni metal foam is Pt sputter coated, some of the Ni is still exposed and dissolves into the water as Ni₂+. In this case, the conductivity of the electrolyte will become higher than that of the DI water used in the other pump versions. Therefore, even if the same power source provided a fixed DC power to these three pumps, a higher current will be induced in the sputtered Pt-coated metal foam pump, which causes a faster flow rate (FIG. 13(b)).

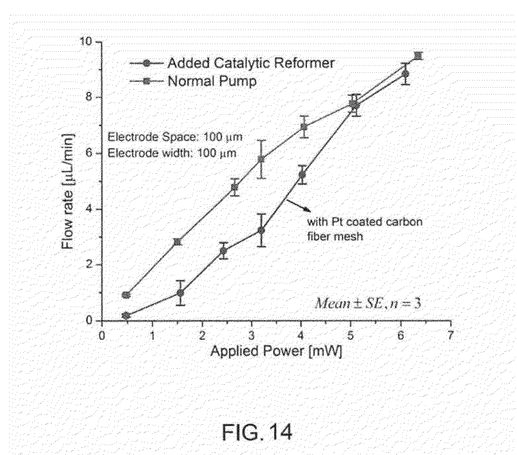

Pt coated carbon fiber mesh is also suitable because carbon is an inert material and will not contaminate the water (electrolyte) during the electrolytic reactions. The same experimental steps were followed for sputtered Pt coated carbon fiber mesh. The corresponding experimental data for flow rate is depicted as FIG. 14. As expected, this kind of catalytic reformer shows a lower flow rate because the contact area between Pt and electrolysis-bubble is much larger, but is still suitable for use herein.

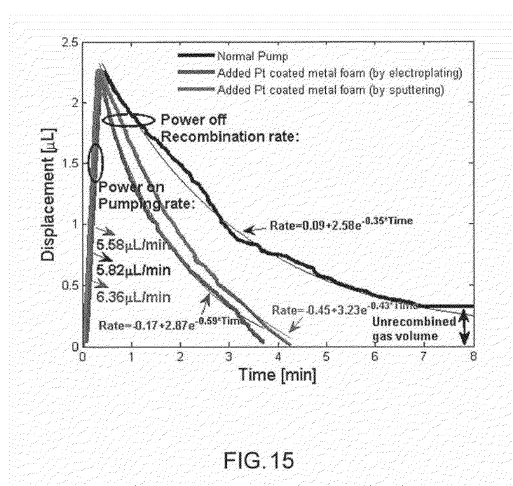

To compare the recombination profiles of the three different pumps, we used Labview's vision assistant to track the red dye flow. As shown in FIG. 15, the electroplated Pt coated metal foam shows a faster recombination rate due to its largest Pt contact area.

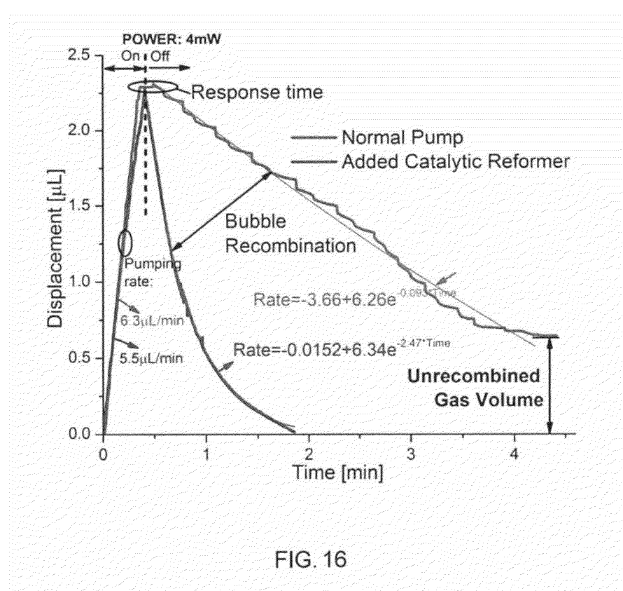

FIG. 16 shows an experimental result about Pt coated carbon fiber mesh's recombination rate. By using this kind of catalytic reformer, the recombination phase becomes faster than the other catalytic reformers. After power was removed, we observed that 72.1% of the pumped volume flows in reverse in the normal pump, illustrating a significant portion of un-recombined gas. While by using our catalytic reformer, 99.3% of the gas gets recombined, showing a great improvement in bubble recombination.

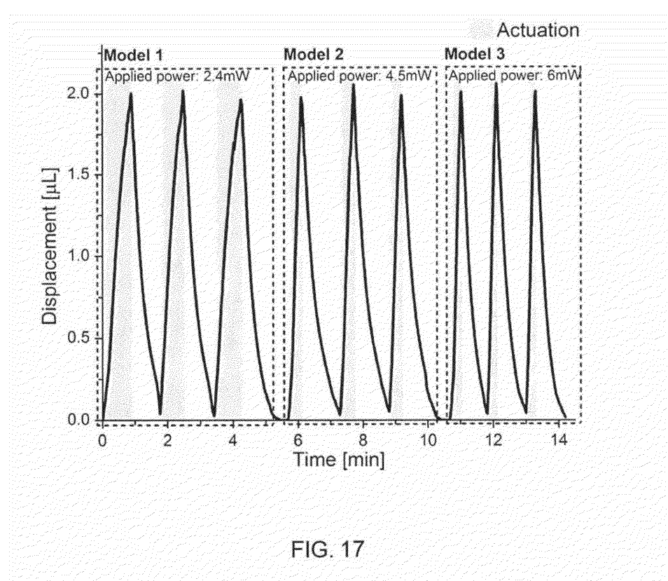

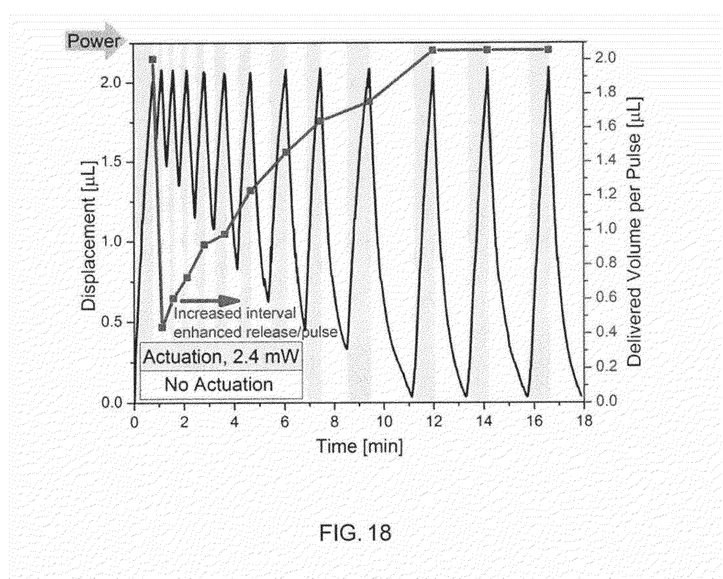

We further selected Pt coated carbon fiber mesh as an example of a catalytic reformer in the following experiments to describe the feasibility of our proposed pump. FIG. 17 illustrates a periodic stable pumping pulse with different power consumptions. By adjusting the applied power, the pumping rate can be controlled; the higher the applied power, the faster flow rate becomes. Actuation frequency is also a factor that can determine the number of pumping cycles and the amount of drug delivered within a given operation period. Our proposed device allows for controllable flow rates as well as an “on-demand” actuation frequency, as per the patient's needs, (as illustrated for example in FIG. 18).

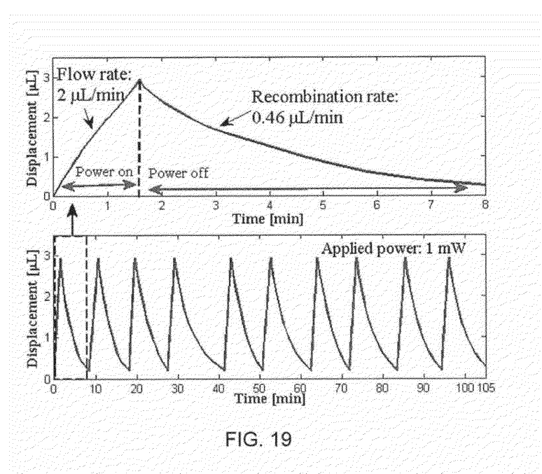

Because our system can involve cyclical pumping to operate, we tested the reverse flow rate during gas recombination, as well as the pump's cyclical operation, as shown in FIG. 19. The bottom plot of FIG. 19 also gives a periodic stable pumping pulse, with relatively low power consumption of 1 mW that definitely can be obtained via a wireless power transfer technique [5].

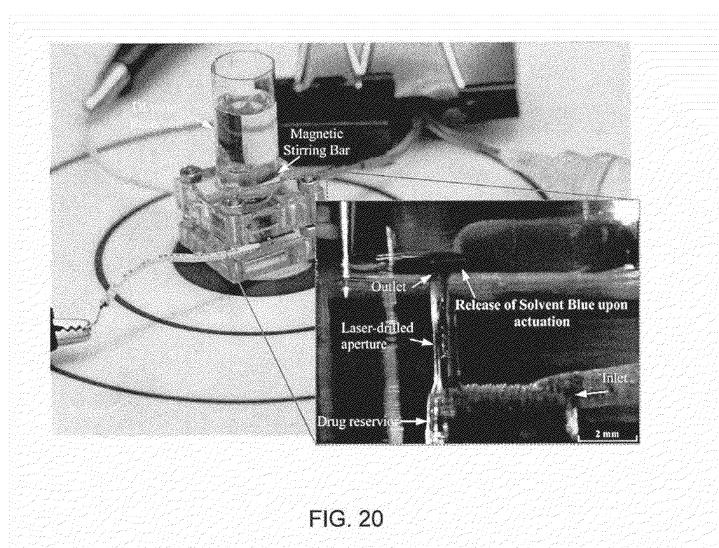

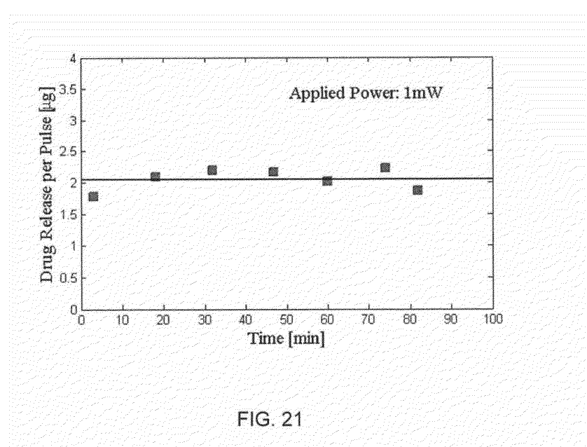

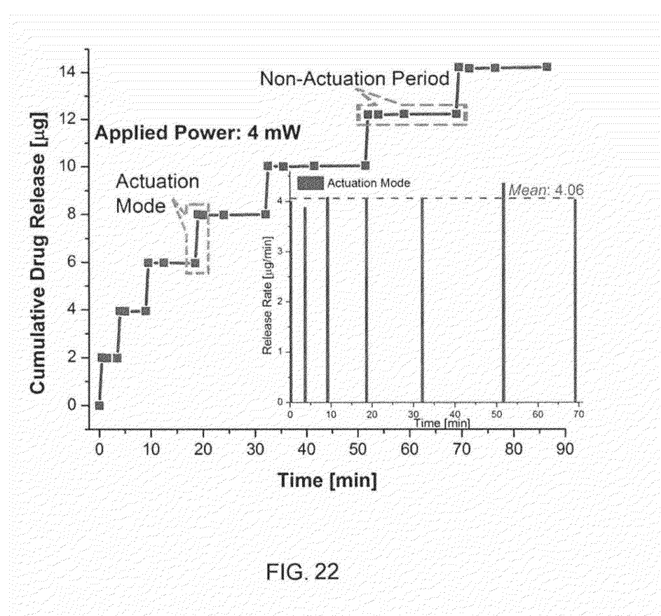

In order to demonstrate the feasibility of our drug delivery device and stable drug release performance by using our proposed pump, a measurement setup is depicted in FIG. 20. We used Solvent Blue 38 (SIGMA) as a low solubility proxy for our solid drug and measured the dose delivered per pulse by extracting the pumped fluid using a pipette and allowing the reservoir to refill with fresh water. The extracted doses were then quantified using a Picodrop Pico200 spectrophotometer and show a stable value of 2 μg/dose over 7 doses as shown in FIG. 21. The cumulative release and drug release rate were obtained as shown in FIG. 22. These results show a release rate of 4.06 μg±0.16 μg/min under intermittent actuations of 4 mW over multi-pulse, which indicate the feasibility and stability of our device.

Thus, our drug delivery system can provide consistent doses from a solid drug stored in a reservoir and together with the integration of our resonant power transfer system can form a drug delivery system that can be flexibly placed within the body and simply controlled without the need for transdermal wiring. Moreover adjusting the applied power can precisely control the pumping of the liquid drug through cyclical bubble generation and recombination.

In various aspects, an electrolytic pump that includes a catalytic reformer in the pumping chamber can improve the cycling time of the electrolytic pump for a drug delivery system. Using our catalytic reforming element, the cyclical actuation of the drug delivery system improved both in terms of the time of the pump/recombination cycle, as well as the applied power requirement, resulting in a faster and more efficient drug delivery system.

In various aspects, the system has been shown to include the following features, among others:

1. An efficiency-optimized layout of electrodes, for example platinum (Pt) electrodes, that can activate electrolysis action and reverse catalytic reaction of the pump.

2. A scaffold, for example a platinum-coated scaffold, can perform as a catalytic reformer for reducing the period of pumping and power requirement.

3. Two PDMS reservoirs can be provided to store an electrolyte (for example, water) and a pumped drug solution, respectively, in order to avoid electrochemical interaction with body fluids.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

### REFERENCES

[1] P. Y. Li, J. Shih, R. Lo, S. Saati, R. Agrawal, M. S. Humayun, Y. C. Tai, and E. Meng, “An electrochemical intraocular drug delivery device,” Sens. Actuators A, Phys., vol. 143, no. 1, pp. 41-48, May 2008.
[2] Li, P. Y., Sheybani, R., Gutierrez, C. A., Kuo, J. T., and Meng, E., “A Parylene bellows electrochemical actuator,” Journal of Micro-electromechanical Systems, vol. 19, no. 1, pp. 215-228, February 2010.
[3] Pirmoradi, F. N, John K. J, Helen M. B, and Mu Chiao. “A magnetically controlled MEMS device for drug delivery: design, fabrication, and testing,” Lab on a Chip 11, no. 18 (2011): 3072-3080.
[4] Sheybani, R, and Ellis M., “High-Efficiency MEMS Electrochemical Actuators and Electrochemical Impedance Spec-troscopy Characterization,” Journal of Microelectromechanical Systems, vol. 21, no. 5, pp. 1197-1208, October 2012.
[5] Y. Yi, U. Buttner, Y. Q. Fan and Ian G. Foulds, “3-Coil Resonance-based Wireless Power Transfer System for Im-plantable Electronic,” In Wireless Power Transfer, 2013 International Conference on, pp. 230-233. IEEE, 2013.
[6] Maruyama, J., Inaba, M., Katakura, K., Ogumi, Z., & Takehara, Z. I. (1998). Influence of Nafion® film on the kinetics of anodic hydrogen oxidation. Journal of Electroanalytical Chemistry, 447(1), 201-209.

We claim at least the following:

1. A drug delivery device, comprising:
a pump and a reservoir associated with the pump,
the pump being an electrolytic pump including a chamber and a plurality of electrodes positioned within the chamber, the plurality of electrodes configured to be coupled to a power supply such that at least one of the electrodes is configured as an anode and at least another of the electrodes is configured as a cathode, the chamber being a sealed chamber configured to hold an electrolyte, the chamber having a side including a flexible impermeable membrane,
the pump further including a reformer positioned within the chamber, the reformer configured to recombine gases generated by the electrolytic pump from an electrolyte contained within the chamber,
the reservoir positioned in association with the membrane of the pump, and
the reservoir configured to receive a drug in solid form and having an orifice configured to allow bodily fluid to pass through the orifice into the reservoir and to allow bodily fluid including the drug to be expelled from the reservoir upon deflection of the membrane.

2. The device of claim 1, wherein the plurality of electrodes comprises an array of electrodes, the array of electrodes formed by a sputtering technique and patterned on a substrate.

3. The device of claim 1, wherein the electrodes are coated with an ionomer that has cationic conductive properties

4. The device of claim 1, wherein the electrodes are platinum electrodes.

5. The device of claim 1, wherein the reformer is a catalytic reformer.

6. The device of claim 1, wherein the reformer is a mesh scaffold.

7. The device of claim 6, wherein the reformer is a platinum-coated mesh scaffold.

8. The device of claim 1, wherein the electrolyte includes water.

9. The device of claim 6, wherein the scaffold is selected from the group consisting of catalytic metals and inert materials

10. The device of claim 9, wherein the catalytic metals include platinum, or nickel or both.

11. The device of claim 9, wherein the inert materials include carbon fiber mesh or a polymer porous mesh or both.

12. The device of claim 1, wherein the reformer is fabricated by sputtering, depositing or electroplating platinum onto a scaffold.

13. A method of drug delivery, comprising the steps of:
providing a pump and a reservoir associated with the pump,
the pump being an electrolytic pump including a chamber and a plurality of electrodes positioned within the chamber, the plurality of electrodes configured to be coupled to a power supply such that at least one of the electrodes is configured as an anode and at least another of the electrodes is configured as a cathode, the chamber being a sealed chamber holding an electrolyte, the chamber having a side including a flexible impermeable membrane,
the pump further including a reformer positioned within the chamber, the reformer configured to recombine gases generated by the electrolytic pump from the electrolyte contained within the chamber,
the reservoir positioned in association with the membrane of the pump, and
the reservoir configured to receive a drug in solid form and having an orifice configured to allow bodily fluid to pass through the orifice into the reservoir and to allow bodily fluid including the drug to be expelled from the reservoir upon deflection of the membrane,
placing a drug in solid form in the reservoir;
using the pump to draw a bodily fluid into the reservoir and dissolve at least some of the solid drug in the bodily fluid;
applying a voltage to the anode electrode and the cathode electrode to thereby generate gas from the electrolyte and generate an increase in pressure within the chamber of the pump causing the membrane to expand and put pressure on the reservoir and causing bodily fluid including the dissolved drug to be expelled from the reservoir;
turning off the applied voltage to the anode and cathode electrodes causing a recombination by the reformer of the gas into the electrolyte thereby causing decrease in pressure within the chamber of the pump causing a decrease in the expansion of the membrane and a resultant pressure drop within the reservoir causing bodily fluid to be drawn into the reservoir; and
repeating one or more cycles of applying voltage and turning off the applied voltage to the anode and cathode electrodes.

14. The method of claim 13, wherein the electrodes are platinum electrodes.

15. The method of claim 13, wherein the reformer is a catalytic reformer.

16. The method of claim 13, wherein the reformer is a mesh scaffold.

17. The method of claim 13, wherein the electrolyte includes water.

18. The method of claim 16, wherein the scaffold is selected from the group consisting of catalytic metals and inert materials

19. The method of claim 18, wherein the catalytic metals include platinum, or nickel or both and the inert materials include carbon fiber mesh or a polymer porous mesh or both.

20. The method of claim 13, wherein the reformer is fabricated by sputtering, depositing or electroplating platinum onto a scaffold.
